(19) United States
(12) Patent Application Publication
(10) Pub. No.: US 2009/0324513 A1
(43) Pub. Date: Dec. 31, 2009

(54) Process for Producing Chlorinated Sucrose

(75) Inventors: Rakesh Ratnam, Mumbai (IN); Shrikant Kulkarni, Mumbai (IN); Suneet Aurora, Mumbai (IN)

Correspondence Address: THE NATH LAW GROUP, 112 South West Street, Alexandria, VA, 22314, US

(73) Assignee: Pharmed Medicare Private Limited, Mumbai (IN)

(21) Appl. No.: 10/593,158

(22) PCT Filed: May 20, 2004
(86) PCT No.: PCT/IN04/00142

§ 371 Date: Sep. 18, 2006

(30) Foreign Application Priority Data
Mar. 19, 2004 (IN) PCT/IN04/00064
May 17, 2004 (IN) 563/MMU/20004

(51) Int. Cl. C07H 5/02; C13K 13/00; A61K 8/72; A61Q 11/00; B32B 1/00; A23G 4/06; A23G 3/36; A23L 2/60; A23L 1/236

(52) U.S. Cl. 424/49; 536/123.13; 428/402; 426/3; 426/658; 426/590; 426/548

(57) ABSTRACT

Present invention relates to disclosure of application of some innovative techniques useful for substantially improving process efficiency of production of chlorinated sucrose including their intermediates and derivatives. Application of mild methods of drying has been made for recovery of chlorinated sucrose or their intermediates and derivatives, in substantially pure form or with other solid chemical impurities, obtained at various stages in the process of production of chlorinated sucrose. Mild methods of drying included agitated thin film drying, spray drying, freeze drying and super critical extraction. Use of alkoxides has been introduced for deacylation instead of alkali hydroxides or alkaline earth hydroxides. Deacylation has been shown to be effective both, either before or after drying of the reaction mixture. Extraction and purification of desired products i.e. of chlorinated sucrose or its intermediates or derivatives, from dried solid mixtures could be achieved by using appropriate extraction method, including but not limited to solvent extraction and super critical extraction. Further purification of such extracts can be done by crystallization or direct drying under mild conditions.

### TECHNICAL FIELD

This invention relates to an improved process for producing chlorinated sucrose

### BACKGROUND OF THE INVENTION

Chloro derivatives derived from sugars, exhibit the organoleptic properties with a very high degree of sweetness compared to the parent sugar. One such chloro sugar prepared from sucrose is 1′,6′-Dichloro-1′,6′-Dideoxy-â-D-Fructo-Furanosyl-4-Chloro-4-Deoxy-â-D-Galactopyranoside. It is a well-known sweetener used widely, including in food and food preparations. Various synthetic routes for the production of 1′,6′-Dichloro-1′,6′-Dideoxy-â-D-Fructo-Furanosyl-4-Chloro-4-Deoxy-â-Galactopyranoside are reported in literature, for e.g. Fairclough et al, Carbohydrate Research 40(1975) 285-298; Mufti et. al. 1983 U.S. Pat. No. 4,380,476; Walkup, et al. 1990 U.S. Pat. No. 4,980,463 and British Patent No. 1543167. They involve preparing derivatives of sucrose, chlorinating them and to recover the desired product, chlorinated sucrose, by reversing derivatization. Preferred processes involve converting sucrose into acetate, chlorination of the acetate, deacylation of the chlorinated acetate and recovering the product from reaction mixture.

A major challenge in these approaches is to separate the desired product, 1′,6′-Dichloro-1′,6′-Dideoxy-â-D-Fructo-Furanosyl-4-Chloro-4-Deoxy-â-D-Galactopyranoside from the chlorinated mass, from other chloro derivatives of sucrose, salts formed during reaction, dark degradation products and tars formed on account of oxidation and elimination due to the relatively severe conditions of chlorination reaction, and finally all these solid components from the large volume of liquids consisting of solvents such as tertiary amide.

Processes for production of this compound reported in patent literature are all based on handling these liquid reaction mixtures by subjecting them, in liquid state itself, to selective liquid-liquid extraction by using organic solvents, column chromatographic purification of the solvent extracts, and the intermediate derivatives and/or the chlorinated sucrose is recovered by conventional crystallization procedure. The procedures used so far for removal of tertiary amides from these liquid reaction mixtures as well as is conventional crystallization are very elaborate, cumbersome to operate and control and expensive on account of the cost of equipment needed and energy required to run the processes.

Navia et al, 1996 U.S. Pat. No. 5,498,709 describes a process for preparing chlorinated sucrose where removal of tertiary amide from the liquid reaction mixture is done by steam distillation. However, the solvent being a high boiling point, it takes a very long time for it to be stripped off to the maximum limits. Also it is reported in the same patent that the volumes of the mass increases to 4 to 5 times of the original volume. This increase in volume also increases the time for isolation of the product as well as increases size of the processing plant to that extent for handling the resultant volume of reactants for further processing.

An improved process, having several potential economic advantages, for preparation of chlorinated sucrose is disclosed in co-pending application No: PCT/IN04/0064. This improved process described recovery of solids, from liquids containing chlorinated sucrose or its intermediates with or without other solids, obtained in a process for producing chlorinated sucrose or otherwise, mainly 1′,6′-Dichloro-1′,6′-Dideoxy-â-D-Fructo-Furanosyl-4-Chloro-4-Deoxy Galactopyranoside by using drying, under mild controlled conditions by industrially used processes, as a more efficient and more effective tool. The described drying process, is applicable to any liquid solution obtained as reaction mixture or purified solution in course of any process including in the course of process for production of chlorinated sucrose or its intermediates. The drying methods which are subject of this co-pending application include, but are not limited to, agitated thin film drying (ATFD), spray drying, freeze drying and super critical extraction. The preferred drying process used in co-pending application was ATFD.

The drying method, as applied in the preceding para has been very effectively used in improving efficiency of operation of post-chlorination steps and recovery of final product intermediate (acetate) and final product in pure solid form. Drying of reaction mixture after completion of chlorination reaction achieved total removal of tertiary amides from the system in a most convenient and most effective way in very short time and better control is obtained on volumes to be handled and effective application of post chlorination steps such as deacylation. Drying also affords a most convenient and more effective and more efficient alternative to batch or continuous processes (some of which described by Navia, et al., U.S. Pat. No. 5,498,706; Mufti, et al., U.S. Pat. No. 4,380,476) of crystallization based on conventional cumbersome crystallization procedures used so far for recovery of final product intermediate or final product from purified liquids obtained in course of production process. The final powder of the product or product intermediate obtained by drying process is amorphous in nature and not crystalline as in conventional crystallization procedure, both having same organoleptic taste and chemical analysis. However, the amorphous powder obtained in the invented process shall have different physical properties such as free-flowing powder properties, different storage properties than the crystalline variety. In that sense, amorphous powder is a new product and its properties are being studied.

### SUMMARY OF INVENTION

In present invention, which is in continuation with the invention disclosed in co-pending application no. PCT/IN04/0064, it was found that efficiency improvement achieved by improvements claimed in the above mentioned co-pending application improves further and becomes more flexible by introducing following alternative routes of further process:
i) Adjusting the pH of the reaction mixture after chlorination to a variety of chosen alkaline ranges, well below pH 11, either neutralization to pH 7.0 to 7.5, or adjusting to pH 7.5 to 9.
ii) Deacylation being done either before or after drying the reaction mixture by drying methods including agitated thin film drying (ATFD).
iii) Use of alkoxides in the steps for deacylation.
   These steps have been very effectively combined with mild methods of drying claimed in PCT/IN04/0064application of the applicants of this patent application to construct very efficient alternative routes for production of chlorinated sucrose having high potential of economic advantages. Some of the alternative routes constructed are as follows:

1. Drying directly the reaction mixture of chlorination step being undertaken of the whole reaction mixture solution either by direct feeding at pH 7.0 to 7.5, or after adjustment of pH to 7.5 to 9.0 or after deacylation step.

2. Drying directly the wash liquid collected from chromatography or any separation method resulting into a purified solution of chlorinated sucrose or its intermediate or derivative for recovery of the product or the product derivative in solid powder form;

3. Dissolving the ATFD dried solids of the chlorination reaction mixture in water, extracting the same in appropriate organic solvent, and drying the solvent extract by ATFD to recover the product or product intermediate in solid powder form;

4. Concentrating the solvent extract obtained in any step of production of chlorinated sucrose, and drying the concentrated extracts to recover the product or product intermediate in solid powder form; or

5. Drying the solution of chlorinated sugar, its derivatives or intermediates obtained otherwise than in the process of production of chlorinated sugars.

The detailed description and the specific examples given in a subsequent section, for the purpose of indicating specific embodiments of the invention, serve the purpose of illustration only. Accordingly, the present invention includes also those various changes and modifications which come within the spirit and scope of the invention that may become obvious to those skilled in the art from this detailed description.

### BRIEF DESCRIPTION OF DRAWINGS AND SHORTFORMS

POCL3 (POCl3) stands for Phosphorous oxy-chloride

ATFD stands for Agitated Thin Film Dryer

TLC stands for Thin Layer Chromatography

HPLC stands for High Pressure Liquid Chromatography

FIG. 1 illustrates the reaction scheme for the preparation of 1′,6′-Dichloro-1′,6′-Dideoxy-â-D-Fructo-Furanosyl-4-Chloro-4-Deoxy-â-D-Galactopyranoside.

FIG. 2 illustrates the agitated thin film dryer used in the process of the present invention,

FIG. 3 is the flow sheet of the agitated thin film dryer;

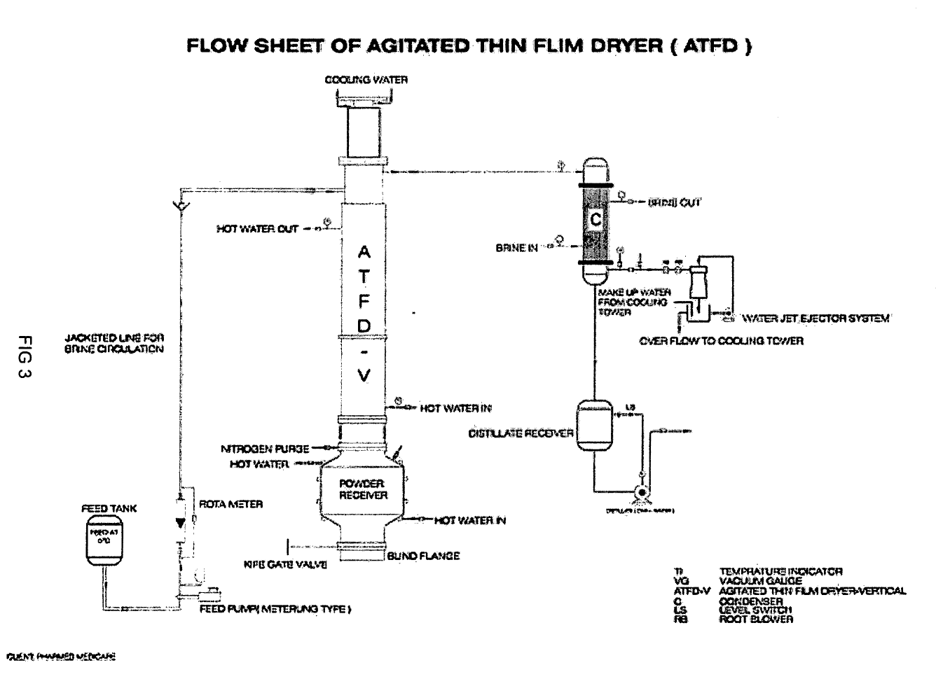

FIG. 4 is the flow chart of the process of the present invention,

FIG. 5 is the IR Report of the product of the present invention; and

FIG. 6 is the HPLC Chromatogram of the product of the present invention.

FIG. 7 is the XRD of crystalline form.

FIG. 8 is the XRD of amorphous form.

### DETAILED DESCRIPTION OF THE INVENTION

It is understood that the customary rules of interpretation of patent documents apply to this document also. However, some of those interpretations, which will expressly apply, are mentioned below.

Present invention is not limited to the particular methodologies, protocols, solvents, and reagents, etc, described herein as tools to achieve the claimed objective, as these may vary with change in context. Further, the terminology used herein is used for the purpose of describing particular embodiments only, and is not intended to and shall not limit the scope of the present invention.

As used herein and in the appended claims, the singular forms “a” “an” and “the” include plural reference also unless the context clearly indicates or requires or means otherwise. Thus, for example, a reference to “a process” is a reference to one or more processes for the stipulated objective and includes equivalents thereof known or obvious to those skilled in the art and so forth. Unless defined otherwise or contrary to the context, all technical and scientific terms used herein have the same meanings as commonly understood by one of ordinary skill in the art to which this invention belongs. Several methods, devices, and materials are described herein, although in practice or for testing of present invention, any methods and materials similar or equivalent to those described herein can be used. All documents cited herein are to be construed for reference and interpretation herein in their entirety

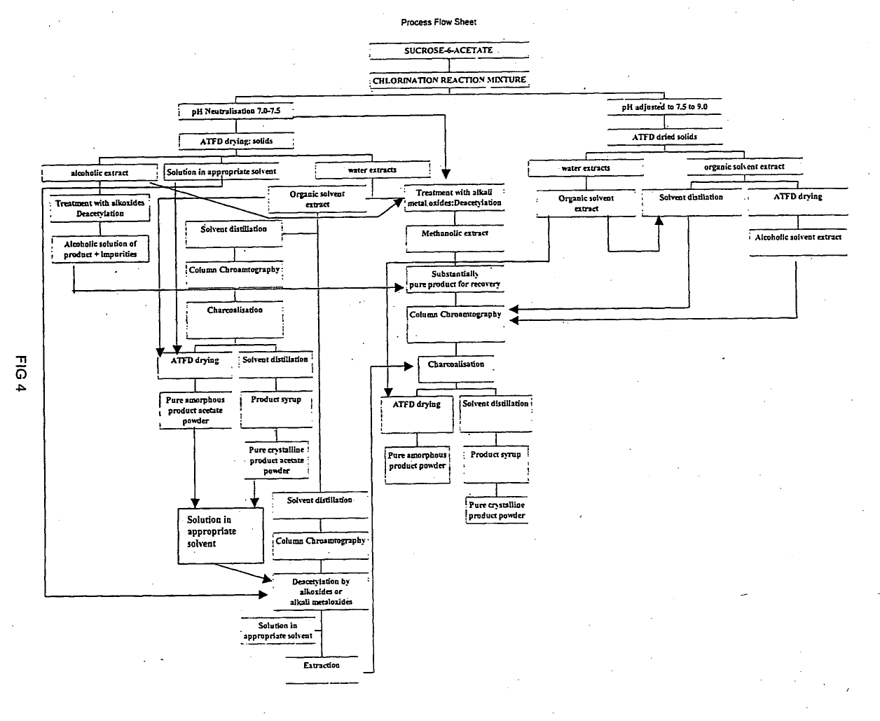

The process of drying under mild conditions applied to any liquid containing chlorinated sucrose derivative, either in substantially pure form or with other accompanying solid ingredients, is illustrated in FIG. 4. Strategic use of some important modifications and the advantages of these modifications and subjecting the liquid solutions subjected to direct drying under mild conditions and certain other modifications introduced in the process, in the context of process efficiency, are as follows:

1. The reaction mixture of chlorination step, after completion of the reaction, can be subjected to drying under mild conditions either after or before deacylation step. This brings in significant improvement in process efficiencies, as it is more easy to handle a dry powder for further purification of the chlorinated sucrose or its intermediate in absence of solvent than in presence of solvent. It is pertinent to mention that the final product as well as inorganic salts formed during the process are both highly soluble in water. Hence, to extract the desired product completely from aqueous medium may be a difficult proposition. However, the extraction of solids (comprising of desired product or its intermediate and inorganic salts) by alcoholic organic solvent is better solution as selective extraction of the desired product will be more easy. The drying process requires less equipment, less consumption of energy, offers freedom from tediousness of crystallization processes in a batch or continuous operation. Several alternative procedures are available to adopt further course of operation either by extracting the chlorinated sucrose or its intermediate either by using a suitable organic solvent, ethyl acetate being preferable, or by supercritical extraction, or by dissolving the solids in water and subjecting the same to purification by column chromatography. Removal of the solvents by drying in above step is more convenient than by steam stripping adopted by Navia et al., in U.S. Pat. No. 5,498,709.

2. Further, once substantially pure solution of chlorinated sucrose or its intermediates or derivatives is obtained in the course of process of their production, recovery of the product is very convenient, direct, total and most effective by resorting to drying under mild conditions than by recovering desired product by conventional crystallization from their concentrated solutions of those products as such or of their derivatives. The process of drying gives substantially pure product directly in one step process in short time with least problems of control without any residual losses of solids; their purity, chemical properties and functional properties such as taste are same as the product obtained by conventional crystallization. This is a decided advantage in view of the cumbersomeness and expenses involved in conventional crystallization and in any process based on conventional crystallization.

3. Such substantially pure solutions of chlorinated sucrose or their intermediates, which could be submitted to drying under mild conditions for recovery of their solids for specific processing advantages, are obtained in the process illustrated in FIG. 4 in the form of wash liquid coming out of chromatographic column when extracts obtained from solids of drying of chlorination reaction mixture are chromatographed and such wash liquid is evaporated and dissolved in appropriate volume of methanol.

In the methods preferred here for the illustration of this invention:

1. The chlorinated sucrose is 1′,6′-Dichloro-1′,6′-Dideoxy-â-D-Fructo-Furanosyl-4-Chloro-4-Deoxy-â-D-Galactopyranoside and also its acetate intermediates.

2. Sucrose-6-ester is sucrose-6-acetate.

3. The method of drying under mild conditions is Agitated Thin Film Drying, which involves evaporation achieved by using a vertical agitated thin film evaporator with hinged rotor blades.

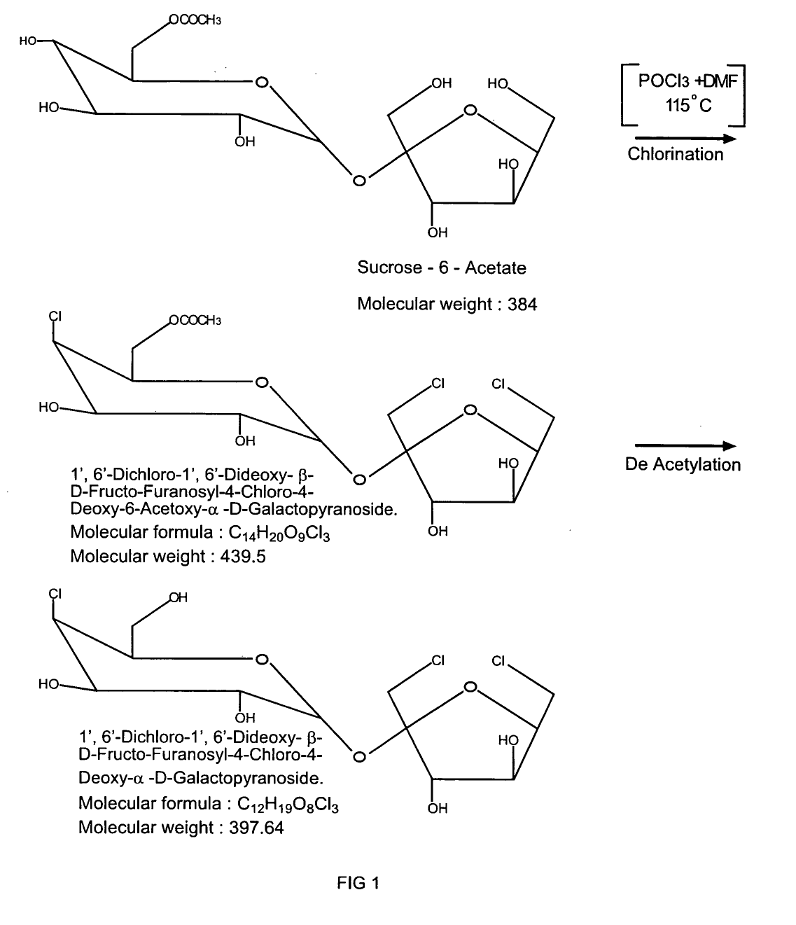

4. The reaction scheme in general involved in the manufacture of product from sucrose-6-acetate is given in FIG. 1 of the accompanying drawings. Many permutations and combinations could be done in the indicated scheme, some of which, but not including all, are shown as illustration in the FIG. 1.

Various operations of the process of production of 1′,6′-Dichloro-1′,6′-Dideoxy-â-D-Fructo-Furanosyl-4-Chloro-4-Deoxy-â-D-Galactopyranoside are as follows:

The sucrose-6-acetate is chlorinated by the Vilsemeier reagent, which is prepared from Phosphorus Oxy Chloride (POCl3) or phosphorus penta chloride (PCl5). The sucrose-6-acetate is added to Vilsmeier Reagent at 5.degree. to 10.degree.C. After completion of the reaction, the reaction mass is heated to 80.degree. to 100.degree.C. and preferably between 90.degree. to 95.degree.C. and maintained for ½-1 hr and then the temperature is raised to 110.degree. to 135.degree.C. and preferably to 120.degree. to 125.degree.C. and maintained for 3-5 hours.

Thereafter the reaction mass is cooled to room temperature and neutralized using alkali hydroxide or carbonate solution.

The desired product 1′,6′-Dichloro-1′,6′-Dideoxy-â-D-Fructo-Furanosyl-4-Chloro-4-Deoxy-â-D-Galactopyranoside or its acetate, obtained after or before deacylation respectively, could be isolated by using the appropriate method mentioned in Methods 1 to 5 given as follows:

Method 1: When the reaction mass after chlorination is directly fed into ATFD, the product obtained was a solid mixture of 1′,6′-Dichloro-1′,6′-Dideoxy-â-D-Fructo-Furanosyl-4-Chloro-4-Deoxy-6-acetoxy-â-D-Galactopyranoside-6-acetate and inorganic salts. The solids were treated with alcoholic solvents and filtered off to remove the inorganics. The alcoholic solution was then heated with appropriate organic alkoxides like sodium methoxide, sodium ethoxide, propoxides or its potassium analogs, during which the deacylation occurs to afford the desired product. The product could be isolated as a solid by either subjecting the alcoholic solution to the ATFD or the alcoholic solution could be further purified by column chromatography and the liquid eluted from the chromatographic column could be subjected to crystallization or direct drying by subjecting to ATFD.

Method 2: The solid mixture of 1′,6′-Dichloro-1′,6′-Dideoxy-â-D-Fructo-Furanosyl-Chloro-4-Deoxy-6-acetoxy-â-D-Galactopyranoside-6-acetate and inorganic salts from the reaction mixture could be dissolved in water or the reaction solution can be further treated directly without ATFD drying with the alkali metal oxides like sodium hydroxide or potassium hydroxide or even with alkali earth metals like calcium hydroxide or barium hydroxide, etc. The resultant solution could be fed into the ATFD or spray drier to remove water and afford solids, where now essentially will be comprising of 1′,6′-Dichloro-1′,6′-Dideoxy-â-D-Fructo-Furanosyl-4-Chloro-4-Deoxy-â-D-Galactopyranoside (the product) and alkali or alkaline earth metal chlorides (NaCl, KCl or CaCl2). The product can be separated from inorganic chlorides by extracting the solids by organic solvents like methanol, ethyl acetate, acetone, etc and the product in the solvent extract can be further purified by column chromatography followed by crystallization or drying in ATFD.

Method 3: Yet another appropriate process was followed by adjusting the reaction mass to pH 7.5-9.0 and then subjecting the reaction mass to ATFD is for solvent stripping. The product obtained being the mixture of desired deacylate of 1′,6′-Dichloro-1′,6′-Dideoxy-â-D-Fructo-Furanosyl-4-Chloro-4-Deoxy-6-acetoxy-â-D-Galactopyranoside-6-acetate and inorganic salts. Effectively the deacylation was achieved in situ during the solvent stripping operations of ATFD. The solids obtained were treated with organic solvents to remove inorganic salts. The crude product after solvent stripping either by spray drying or ATFD was further purified after extracting solids in alcoholic solvents by column chromatography followed by crystallization or subjecting the chromatography column eluate to ATFD drying.

Method 4: Isolation of the pure 1′,6′-Dichloro-1′,6′-Dideoxy-â-D-Fructo-Furanosyl-4-Chloro-4-Deoxy-6-acetoxy-â-D-Galactopyranoside-6-acetate from solids containing 1′,6′-Dichloro-1′,6′-Dideoxy-â-D-Fructo-Furanosyl-4-Chloro-4-Deoxy-6-acetoxy-â-D-Galactopyranoside-6-acetate and inorganic salts in a process mentioned in methods 1 and 2 could also be achieved by direct column chromatography of the salts obtained from ATFD or spray drier.

Method 5: Isolation of the pure 1′,6′-Dichloro-1′,6′-Dideoxy-â-D-Fructo-Furanosyl-4-Chloro-4-Deoxy-6-acetoxy-â-D-Galactopyranoside-6-acetate from solids containing 1′,6′-Dichloro-1′,6′-Dideoxy-â-D-Fructo-Furanosyl-4-Chloro-4-Deoxy-6-acetoxy-â-D-Galactopyranoside-6-acetate and inorganic salts could also be achieved by dissolving the solids with water and extracting with organic solvents like dichloromethane, ethyl acetate and then stripping of solvent by drying by ATFD to get crude solid mixture. Isolation of the pure 1′,6′-Dichloro-1′,6′-Dideoxy-â-D-Fructo-Furanosyl-4-Chloro-4-Deoxy-6-acetoxy-â-D-Galactopyranoside from solids containing 1′,6′-Dichloro-1′,6′-Dideoxy-â-D-Fructo-Furanosyl-4-Chloro-4-Deoxy-6-acetoxy-â-D-Galactopyranoside-6-acetate can be achieved after followed by deacylation as mentioned in method 1 and 2.

Method 6: Isolation of the product 1′,6′-Dichloro-1′,6′-Dideoxy-â-D-Fructo-Furanosyl-4-Chloro-4-Deoxy-â-D-Galactopyranoside from solids containing 1′,6′-Dichloro-1′,6′-Dideoxy-â-D-Fructo-Furanosyl-4-Chloro-4-Deoxy-â-D-Galactopyranoside and inorganic salts could be done by dissolving the solids obtained by using process described in Method 3 in water and extracting the product in organic solvent like ethyl acetate, dichloromethane, etc. This was followed by solvent stripping, column chromatography and crystallization.

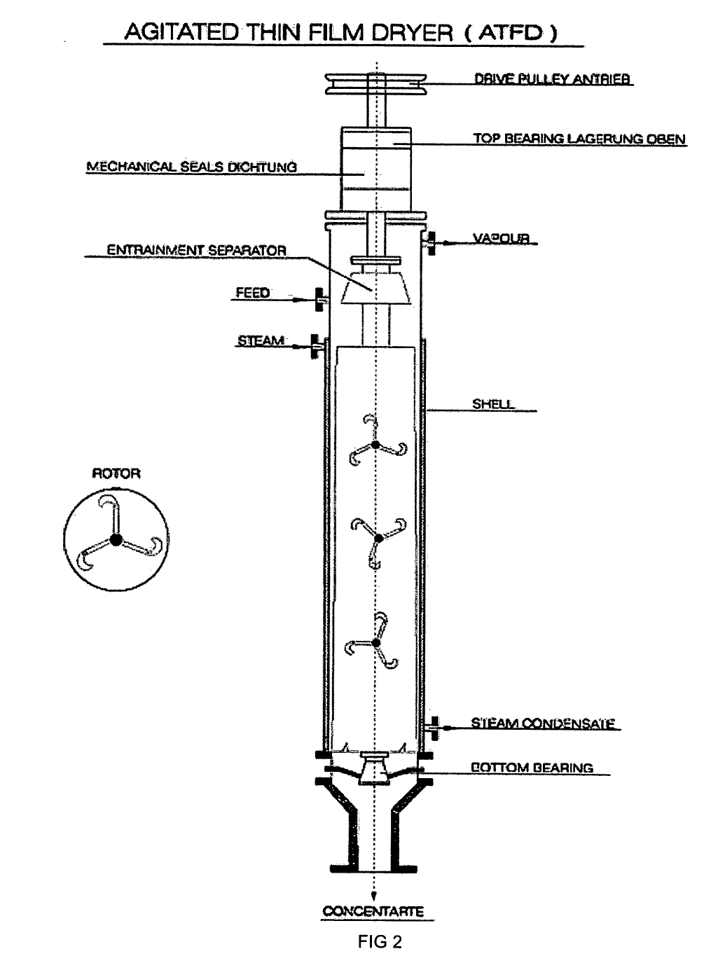

Diagram of ATFD is shown in FIG. 2 of the drawings.

The detailed preferred process of ATFD is illustrated as follows:

Feed of the reaction mass was cooled to 5.degree. to 10.degree.C. in the feed tank by circulating a brine solution. A pump was used to lift the feed from feed tank to the Dryer. The ATFD is a vertical Dryer with area of cross section 0.25 to 0.35 meter square. The feed enters tangentially and spreads along the inside surface of the shell in to a thin film. The rotor blades are hinged; the hinged rotor blades keep the film under intense agitation preventing any scale formation. The speed of the rotor was 1000 to 1500 revolutions per minute. The film progressively passes through different phases like liquid, slurry, paste, wet powder and finely powder of desired dryness, it is collected in a powder receiver.

The vapor flows countercurrent to the solids and was removed from the top of the Dryer. Distillate was collected from the condenser and solids are obtained from the Dryer. The distillate contains solvent and water. The distillate was subjected to fractional distillation, about 70-80% of solvent was recovered based on the input of solvent.

### EXPERIMENT

1. Chlorination of Sucrose-6-acetate

100 g of sucrose-6-acetate was mixed with 200 ml of fresh solvent such as hexane, cyclohexane, pyridine, dimethyl formamide, and others, and particularly dimethyl formamide and Chlorination undertaken in a 3 liter 3 neck round bottom flask. 500 ml of the solvent was charged. Thereafter, the solvent was cooled with stirring to 0.degree. to 5.degree.C. To this reaction mass, 166 ml of phosphorous oxy chloride (273.9 g) was added below 0.degree.C. To this chlorinating reagent, 100 grm of sucrose 6-acetate in solvent was added below 10.degree.C. Thereafter, the reaction mass was stirred at 20.degree. to 25.degree. C. for ½-1 hr. Further, the temperature was raised to 70.degree. to 100.degree. to C. and preferably 80.degree. to 90.degree.C. and was maintained for 1 to 2 hr. Afterwards the temperature was raised to 110.degree. to 130.degree.C. and preferably 120.degree. to 122.degree.C. and was maintained for 3 to 5 hr. The reaction mass was cooled to 40.degree. to 45.degree.C. and was neutralized.

### 2. ATFD Removal of Solvent

The reaction mass obtained after completion of chlorination reaction, which approximates volume of 2-2.3 liter, was further treated in one of the following, including but not limited to, two alternatives:
a) pH is adjusted to 7.0-7.5 to obtain mixture of 1′,6′-Dichloro-1′,6′-Dideoxy-â-D-Fructo-Furanosyl-4-Chloro-4-Deoxy-6-acetoxy-â-D-Galactopyranoside-6-acetate and inorganic salts;

b) pH is adjusted to 7.5-9.0 to obtain mixture of 6-acetyl intermediate of 1′,6′-Dichloro-1′,6′-Dideoxy-â-D-Fructo-Furanosyl-4-Chloro-4-Deoxy-â-D-Galactopyranoside and inorganic salts;

and the reaction mass was then fed to ATFD with the parameters, which are described, but not limited to, as follows:.

Area of ATFD=0.20-0.50 meter square

Feed rate=7-10 kg/hr

Pressure=2-10 mm Hg.

Jacket temp=70.degree. to 100.degree.C.

Feed of 3 to 5 kg was cooled down to 5.degree. to 10.degree.C. in the feed tank by circulating the brine solution. pH of the feed was maintained at 7.0 to 7.5 or 7.5 to 9; the pump was fitted to lift the feed from feed tank to the dryer. The dryer is a vertical dryer with area of cross section 0.25-0.35 square Meter. The feed entered tangentially and spreads along the inside surface of the shell in to a thin film. The rotor blades are hinged, the hinged rotor blades keep the film under intense agitation preventing any scale formation. The speed of the rotor was 1000-1500 revolutions per minute. Temperature was maintained around 70.degree. to 100.degree.C. in the jacket by circulating hot water taking inlet from bottom, outlet through the top. The film progressively passed through different phases like liquid, slurry, paste, wet powder and finely powder of desired dryness. This was collected in a powder receiver.

The dry product, which was essentially, a mixture of 1′,6′-Dichloro-1′,6′-Dideoxy-â-D-Fructo-Furanosyl-4-Chloro-4-Deoxy-6-acetoxy-â-D-Galacto-pyranoside-6-acetate and inorganic salts or alternatively deacylated 1′,6′-Dichloro-1′,6′-Dideoxy-â-D-Fructo-Furanosyl-4-Chloro-4-Deoxy-6-acetoxy-â-D-Galactopyranoside-6-acetate and inorganic salts. This depends on the respective feed to the ATFD as described above.

The vapor flows countercurrent to the solids and was removed from the top of the Dryer. These vapors were condensed in the condenser. Distillate was collected from the condenser, solids were obtained from the Dryer. The distillate contains solvent and water.

The distillate was subjected to fractional distillation, about 70-80% of solvent was recovered based on the input of solvent.

3. Recovery of 1′,6′-dichloro-1′,6′-dideoxy-â-D-fructo-furanosyli-4-chloro-4-deoxy-â-D-galctopyranoside from 6-acetyl Intermediate, Its Deacylation and Recovery of Final Product:

This was achieved in four alternative ways exemplified as follows:

3.1 The pH of the reaction mass after chlorination was adjusted to 7.0-7.5. The reaction mass was subjected to ATFD to obtain 450 g of mixture of 1′,6′-Dichloro-1′,6′-Dideoxy-â-D-Fructo-Furanosyl-4-Chloro-4-Deoxy-6-acetoxy-â-D-Galactopyranoside-6-acetate and other inorganic salts. This powder was dissolved in 1.2 liters of water and complete dissolution was ensured. The solution was then extracted with 1 liter of ethyl acetate and layers separated. The aqueous solution was re-extracted with 400 ml of ethyl acetate three times and all the ethyl acetate layers were pooled. The ethyl acetate was distilled off and the residue obtained was purified by column chromatography on silica gel. The purified 6-acetyl intermediate was deacylated with 10% sodium methoxide solution in methanol (pH 9-10).

The deacylated product was concentrated and crystallized to obtain pure 1′,6′-dichloro-1′,6′-Dideoxy-â-D-Fructo-Furanosyl-4-Chloro-4-Deoxy-â-D-Galactopyranoside of 98.6% purity by HPLC and yield 30%.

3.2 450 g solids obtained from the ATFD which contains the 6-acetyl intermediate of 1′,6′-Dichloro-1′,6′-Dideoxy-â-D-Fructo-Furanosyl-4-Chloro-4-Deoxy-â-D-Galactopyranoside and the inorganic salts were dissolved in 500 ml methanol three times. The methanolic extract was filtered to remove the inorganic salts.

The methanolic solution containing 6-acetyl product was treated with sodium methoxide at room temperature and was mixed till complete deacetylation. The product was then concentrated to thick syrup and was taken for silica gel column chromatography. The purified fractions were collected, concentrated and crystallized to get pure product of 98.9% and yield 34%.

3.3 The pH of the chlorinated mass was adjusted to 8.5-9.0 and was fed to the ATFD. Solids obtained from ATFD, 450 g, were extracted into 500 ml methanol three times. The methanol extract was filtered and concentrated and the residue was purified by silica gel column chromatography. The product obtained was 92% pure.

3.4 Solids from ATFD of the step of drying in example 3, 450 g, were dissolved in 1.2 liter water and then extracted into 1:1 ethyl acetate. The aqueous solution was further extracted with 400 ml of ethyl acetate thrice. The ethyl acetate layers were pooled together and concentrated. The thick syrup obtained was purified by column chromatography to yield pure product of 93%.

### 4. Chromatographic Purification and Charcoalization

Purification of liquids containing product or product intermediates with impurities was purified by column chromatography on silica gel or alumina using appropriate elution/desorbtion medium. The eluent coming out of the column was given charcoal bed treatment and was then sent for either ATFD drying or for conventional crystallization.

### 5. Extraction of Product From Solids Obtained After ATFD Drying of Purified Liquids Containing the Product

The solid mass obtained from the ATFD, when the liquid subjected to drying contained almost pure product, was subjected to solvent extraction. The solvent used may be any organic solvent, including but not limited to, ethyl acetate, methanol, methyl ethyl ketone, and acetone. The preferred solvent used was methanol.

The solvent extracted mass was distilled in the rotary evaporator at low temperature. The syrup obtained was mixed with an appropriate column chromatography adsorbent like silica gel or alumina and run through column chromatography. The adsorbing agent could be any known column packing preferably alumina or silica gel. The preferable solvents for desorbption are ethyl acetate, mixture of toluene and methanol, mixture of methanol and ethyl acetate, mixture of methanol and dichloromethane. Ethyl Acetate was used in this work. The eluted fractions were collected in different receivers based on TLC showings. The fractions showing single spot on the TLC were collected separately. The solvent from this fraction was evaporated to provide a thick syrup. The thick syrup was subjected to purification. Crude product obtained showed by TLC to have a high concentration of the desired product. This was subjected to crystallization.

The products that are extracted and purified by the above processes were and could be any one of the following i.e. 1′,6′-Dichloro-1′,6′-Dideoxy-â-D-Fructo-Furanosyl-4-Chloro-4-Deoxy-6-acetoxy-â-D-Galactopyranoside-6-acetate or its deacylated form i.e. 1′,6′-Dichloro-1′,6′-Dideoxy-â-D-Fructo-Furanosyl-4-Chloro-4-Deoxy-6-acetoxy-â-D-Galactopyranoside.

### 6. Isolation of Product Acetate or the Product in Powder Form

The syrup obtained from the isolation stage was mixed with 3-5 volumes of mass of ethyl acetate and mixed well. The mass was distilled at low temperature to remove 2-4 volumes of ethyl acetate resulting in a liquid product concentrate. The desired dry product was obtained from the liquid concentrate by three methods.

a) Conventional crystallization, as reported as in the earlier patents cited in this document. Essential steps consisted of solvent distillation to form a product syrup, which was then dissolved in minimum quantity of appropriate solvent and then seeded with the product crystals for crystallization. The pure Crystals were recovered by centrifugation.

b) Feeding liquid product concentrate to ATFD to obtain the dry desired pure product of the acetyl intermediate or the desired deacylated product. This has been reported for the first time in the co-pending application of these applicants No. PCT/N04/D064.

c) Spray drying the liquid product concentrate to obtain of the acetyl intermediate or the desired deacylated product, which has also been reported for the first time in the co-pending application of these applicants No. PCT/N04/D064.

In the conventional crystallization method the liquid concentrate was crystallized to obtain solid product. The product was filtered and dried under vacuum at 40.degree. to 50.degree. C.

### 7. Properties of the Product Obtained Through Above Experiments

The solids isolated after drying the purified product containing liquids in the ATFD were also found to be identical in taste, organoleptically, and chemical analysis with the product obtained from the conventional crystallization method. Also the solids obtained after spray drying the liquid concentrate were found to be identical in taste and chemical purity with the desired pure product obtained from the crystallization and ATFD method. Solid powders obtained by ATFD and other methods of drying when compared to powders obtained from crystallization procedures were, however, amorphous in nature having smaller particle size. The average particle size of chlorinated sucrose, its derivatives and its intermediates was observed to be less than 20 microns, average particle size within a range of 5 micron to 12 microns, the residual moisture content was less than 10%, more particularly to less than 5% and usually less than 0.5% up to 0.3%. This small particle size is obtained directly in crystallization or precipitation procedure and is not result of any milling after the crystals are obtained. The powder which appears as amorphous may also a microcrystalline in nature and composed of full range of particle shapes from totally amorphous though globular shapes to well defined needles. The product may further be milled to achieve more uniform particle size distribution. Residual solvent content of the product produced by the process describe above was below 0.1% usually 0.09%.

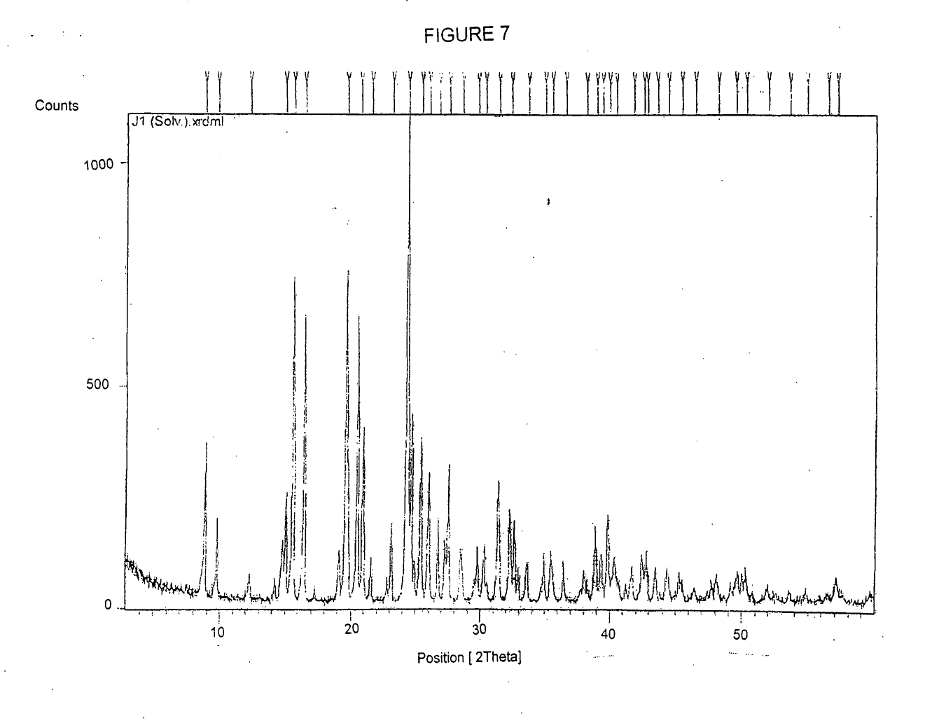

The crystalline form of the product is confirmed with the XRD plot as shown in FIG. 7.

The particle size was evaluated in Microtrac—X100 equipment.

Properties of some of the batches of products are given below:

TABLE 1
Properties of conventional crystallization batch
in process described in experiment no. (6.a):

| SI. | | | |
|---|---|---|---|
| No | TEST | RESULTS | SPECIFICATION |
| 01 | Appearance | White coloured | White to off white |
| | | crystals, taste sweet. | crystals, taste sweet. |
| 02 | Solubility | Complies | Soluble in ethanol and |
| | | | water. Slightly soluble |
| | | | in ethyl acetate. |
| 03 | Particle size | | 90% ≦ 9 microns |
| 04 | Specific Rotation | +86.03° | Between +84° to |
| | | | +87.5° |
| 05 | Water content | 0.22% | NMT 2% |
| 06 | Residue on | 0.04% | NMT 0.7% |
| | ignition | | |
| 07 | Identification by | Complies | Complies with |
| | IR | | standard. |
| 08 | Identification by | Complies | Rf should complies |
| | TLC | (Rf of both std & | with std |
| | | sam = 0.72 cm) | |
| 09 | Other chlorinated | Complies | NMT 0.5% |
| | disaccharides | | |
| 10 | Chlorinated | Complies | NMT 0.1% |
| | monosaccharides | | |
| 11 | Triphenylphosphine | Complies | NMT 150 μg/g |
| | oxide | | |
| 12 | Methanol | 0.09% | NMT 0.1% |
| 13 | Organoleptic | Passes the test | Passes the test |
| 14 | Purity | 99.65% | NMT 98% |
| 15 | Arsenic | Complies | NMT 3 ppm |
| 16 | Heavy metals | Complies | NMT 0.001% |

MICROBIOLOGICAL SPECIFICATIONS

| | | | |
|---|---|---|---|
| 21 | Total aerobic | Nil | NMT 250/g |
| | count | | |
| 22 | Yeasts | Nil | NMT 50/g |
| 23 | Molds | Nil | NMT 50/g |
| 24 | Coliforms | Nil | Negative test |
| 25 | E. Coli | Nil | Negative test |
| 26 | S. aureus | Nil | Negative test |
| 27 | Salmonella | Nil | Negative test |

TABLE 2
The table below gives the data with respect to the
individual crystal particle and their percentage

| | |
|---|---|
| 10% | 0.956 |
| 20% | 1.541 |
| 30% | 2.093 |
| 40% | 2.605 |
| 50% | 3.274 |
| 60% | 4.236 |
| 70% | 5.311 |
| 80% | 6.615 |
| 90% | 9.185 |
| 95% | 11.51 |

It is conclusive from the above results that the particle size of 90% of the crystallized product is below 9.185 microns.

TABLE 3
Summary results on particle size obtained from some other batches

| Batch No. | Particle size |
|---|---|
| PP 02 | 90% ≦ 9.125 microns |
| PP 03 | 90% ≦ 9.165 microns |
| PRO 01 | 90% ≦ 9.035 microns |
| PRO 05 | 90% ≦ 9.183 microns |

The particle size of crystals thus obtained from ethyl acetate crystallization conforms to the specifications.

Properties of amorphous product obtained by direct drying methods such as ATFD and spray drying:

The average particle size of chlorinated sucrose, its derivatives and its intermediates as was obtained from ATFD and or spray drying was observed to be less than 15 microns, average particle size within a range of 5 micron to 12 microns, the residual moisture content was less than 10%, more particularly to less than 5% and usually less than 0.5% up to 0.3%. This small particle size is obtained directly after the said drying process and is not subjected to any milling after the dried powder is obtained. Also the spray drying or ATFD can be carried out on the pure product as well as in mixture with other suitable diluents or formulating agents. The powder which appears as amorphous may contain solvent content of below 0.1% usually 0.09%.

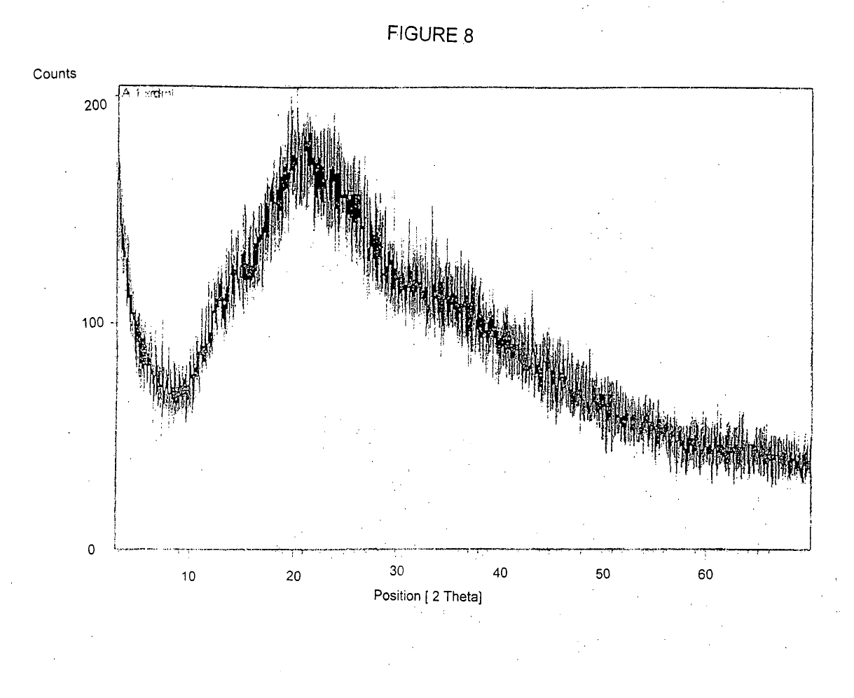

The XRD plot as shown in the FIG. 8 indicates no regular pattern, without any peaks, confirming that the product is amorphous in nature.

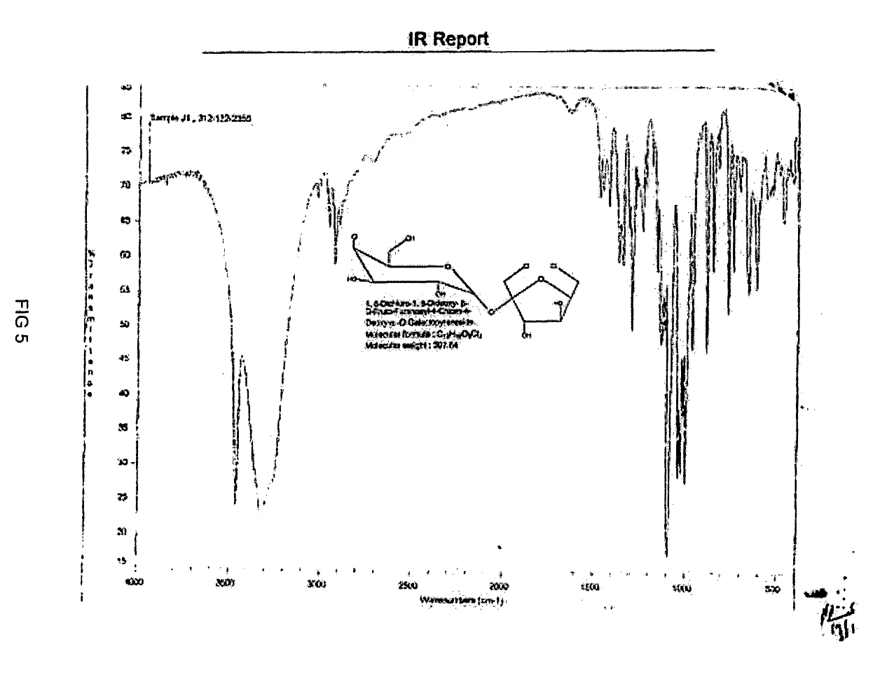

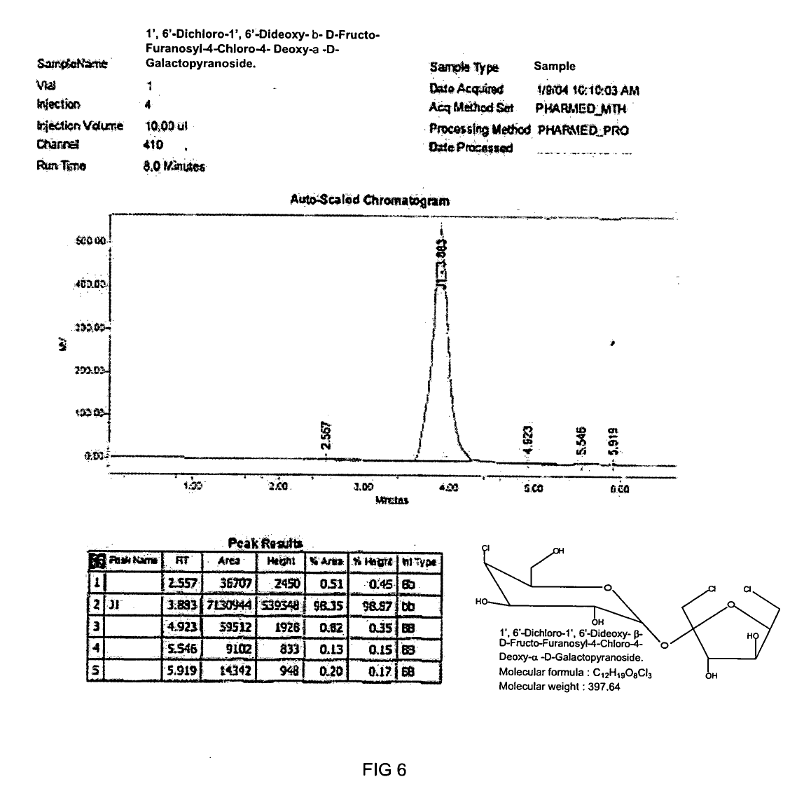

The chemical analysis of the solids from all the three methods showed the product purity or content was over 99% (HPLC FIG. 6 and IR FIG. 5 attached).

In a separate experiment, the ethyl acetate extract from after column chromatography was directly passed through ATFD. This also resulted in a desired pure product of high purity.

The solid product could be also isolated by feeding the ethyl acetate extract after column chromatography directly into the Spray Dryer or any other Dryer to afford the solid product.

1-25. (canceled)

26. A process of handling solution of sucrose intermediates and derivatives, including, chlorinated sucrose, comprising:
a) removal of liquids from the said solution by direct drying, under conditions mild enough to prevent degradation or modification of chlorinated sucrose, for recovery of solids from the said liquids and the end product of such operations is a solid mass of the chemicals visibly free from the said liquid;
b) recovering the said solids, present in the said liquid either in substantially pure form or with other solid impurities;
c) the said liquids being obtained in a process of producing chlorinated sucrose, mainly 1′,6′ Dichloro-1′,6′-Dideoxy-â-D-Fructo-Furanosyl-4-Chloro-4-Deoxy-â-D-Galactopyranoside;
the said method of drying including one or a combination of, agitated thin film drying, spray drying, freeze drying and super critical extraction.
wherein the process of production of chlorinated sucrose comprises of,
i) deacylation of intermediates of chlorinated sucrose before as well as after drying of the chlorination reaction mixture by mild drying methods described above;
ii) use of alkali metal oxides as well as alkoxides, including Potassium Methoxide or Sodium Methoxide, for deacylation;
iii) achieving deacylation up to pH of 9 but well below pH 11.

27. The process of claim 26, wherein the chlorinated sucrose (or its
intermediates or derivatives) containing liquid is a mixture of the respective substantially pure forms as well as of several solid ingredients of other chemicals in dissolved or suspended state.

28. The process of claim 27 wherein the individual ingredients of the said mixture of solids, containing chlorinated sucrose (or its intermediates or derivatives) as one of the ingredients, originate from reactants of a process undertaken for chlorination of sucrose-6-esters.

29. The process of claim 28 wherein the sucrose-6-ester is sucrose-6-acetate or sucrose-6-benzoate.

30. The process of claim 29 wherein the chlorinating reagent is any one suitable for chlorinating sucrose-6-ester.

31. The process of claim 30 wherein the said chlorinating reagent is a Vilsmeier reagent of the formula [XCIC═NR₂]⁺Cl⁻ (where R represents an alkyl group and X represents a hydrogen atom or a methyl group].

32. The process of claim 28 wherein in the said process of chlorination, sequence of steps involves addition of sucrose-6-ester solution in a tertiary amide to the chlorinating reagent for chlorination.

33. The process of claim 32 wherein the said tertiary amide is N,N-dialkylformamide.

34. The process of claim 33 wherein the said N,N-dialkylformamide is dimethylformamide.

35. The process of claim 26, wherein the chlorinated sucrose containing liquid contains chlorinated sucrose in pure form with impurities in small or trace quantities.

36. The process of claim 35 wherein the said chlorinated sucrose containing liquid, is a wash solvent collected as effluent from a column chromatography of an impure solution of chlorinated sucrose.

37. The process of claim 36 wherein the said wash solvent is subjected to concentration before subjecting to drying treatment.

38. The process of claim 36 wherein the said wash solvent used for desorbtion is either a single solvent like ethyl acetate, or mixture of solvents like mixture of toluene and methanol or mixture of methanol or water & ethyl acetate.

39. The process of claim 36 when the said column chromatography is done by using a suitable adsorbent preferably, alumina or silica gel.

40. The process of claim 36 when the said impure solution is the crude extract of chlorinated sucrose (or its intermediates or derivatives) from a solid powder mixture of several chemicals, including chlorinated sucrose; extraction being done by any suitable extraction process including supercritical extraction or by conventional extraction in any suitable solvent including water, ethyl acetate, methanol, methyl ethyl ketone, acetone, which are capable of selective extraction of substantially pure form of chlorinated sucrose free from impurities.

41. The process of claim 37 wherein the concentrated extract is subjected to conventional crystallization for purification of chlorinated sugar.

42. The process of claim 28, wherein the said process of chlorination comprises of:
i) preparation of Vilsmeir reagent from Phosphorus oxy-chloride,
ii) addition of sucrose-6-ester, preferably sucrose-6-acetate, to Vilsmeier reagent at 5° to 10° C. and allowing reaction to complete,
iii) heating the reaction mixture to 80° to 100° C., preferably between 90° to 95° C. and maintained for half to one hour,
iv) raising temperature of reaction mixture of step no. (iii) to 110° C., preferably to 120.° to 130° C. and maintained for 3-5 hours,
v) cooling the reaction mass to room temperature, cooling the reaction mass into a solution of a suitable deacylating reagent in inorganic basic solution like alkali hydroxide solution accompanied by further cooling to keep the temperature below 30° to 35° C.,
vi) adjusting the pH to 7 to 9.5 and preferably 8-9.

43. The process of claim 42 wherein at step no. v), wherein any alkoxide, preferably Potassium Methoxide or Sodium Methoxide is used instead of alkali metal oxides for deacylation.

44. The process of claim 42 wherein pH is adjusted only up to 9 and reaction mixture is subjected to removal of liquids from the said solution by direct drying, under conditions mild enough to prevent degradation or modification of chlorinated sucrose, for recovery of solids from the said liquids and the end product of such operations is a solid mass of the chemicals visibly free from the said liquid.

45. The process of claim 26 wherein the solids obtained from drying of reaction mixture from chlorination step are extracted for chlorinated sucrose recovery by any suitable method of extraction, including, solvent extraction.

46. The process of claim 36 wherein the said impure solution is the solution of the solid powder mixture of several chemicals, including chlorinated sucrose, made in water and subjected to purification by application of separation methods including column chromatography, extraction in water immiscible solvent having selective affinity with chlorinated sucrose or chlorinated sucrose intermediates or chlorinated sucrose derivatives

47. The process of claim 36 when the said impure solution is the crude extract of chlorinated sucrose (or its intermediates or derivatives) from a solid powder mixture of several chemicals, including chlorinated sucrose; extraction being done by water and the water extract being subjected to a any suitable extraction process including to conventional extraction in any suitable solvent, including ethyl acetate, methanol, methyl ethyl ketone, acetone, which are capable of selective extraction of substantially pure form of chlorinated sucrose free from impurities.

48. A solid powder form of a chlorinated sucrose, its intermediates, its derivatives of process of claim 26, at a least part of which is amorphous or non crystalline.

49. Chlorinated sucrose, its intermediates, its derivatives of claim 48 which comprises of:
i) average particle size of 8 micron or less, within a range of 5 micron to 8 micron.
ii) residual moisture content of 10% or less, more particularly less than 5%, still more particularly less than 0.5%.

50. Chlorinated sucrose, its intermediates, its derivatives of chlorinated sucrose, its intermediates, its derivatives, at least a portion of which comprises of particles less than 20 micron precipitated as microcrystalline particles directly from a process of crystallization.

51. Chlorinated sucrose, its intermediates, its derivatives of claim 50 which comprises of:
i) average particle size distribution of 12 micron or less, majority of particles being within a range of 8 micron to 10 micron
ii) various shapes ranging from globular particles to fully crystallized needles
iii) residual moisture content of 10% or less, more particularly less than 0.5%, still more particularly less than 0.3%

52. Chlorinated sucrose, its intermediates, its derivatives at least a part of which consists of amorphous or non crystalline or of particles less than 20 micron microcrystalline particles produced directly from a process of crystallization.

53. An oral composition, ingestible as well as non-ingestible including a toothpaste and a chewing gum, a food, a beverage; high intensity sweetener composition; in solid, semi-solid or liquid form, to which is added a composition of chlorinated sucrose of claim 48.
